(19) United States
(12) Patent Application Publication
(10) Pub. No.: US 2010/0174340 A1
(43) Pub. Date: Jul. 8, 2010

(54) Methods and Apparatus for Applying Energy to Patients

(75) Inventor: Bruce J. Simon, Mountain Lakes, NJ (US)

Correspondence Address: ELECTROCORE INC., 51 GILBRALTAR DRIVE, SUITE 2F, POWER MILL PLAZA, MORRIS PLAINS, NJ, 07950-1254, US

(73) Assignee: ElectroCore, Inc., Morris Plains, NJ (US)

(21) Appl. No.: 12/652,302

(22) Filed: Jan. 5, 2010

Related U.S. Application Data
(63) Continuation-in-part of application No. 12/246,605, filed on Oct. 7, 2008.
Application No. 12/246,605 is a continuation-in-part of application No. 11/735,709, filed on Apr. 16, 2007.
Application No. 11/735,709 is a continuation-in-part of application No. 12/422,483, filed on Apr. 13, 2009.
Application No. 12/422,483 is a continuation-in-part of application No. 12/408,131, filed on Mar. 20, 2009.
(60) Provisional application No. 60/978,240, filed on Oct. 8, 2007.
(60) Provisional application No. 60/792,823, filed on Apr. 18, 2006.

(51) Int. Cl. A61N 1/36; A61N 1/05

(52) U.S. Cl. 607/40; 607/116; 607/46

(57) ABSTRACT

The present invention provides systems, apparatus and methods for selectively applying electrical energy to body tissue. More specifically, systems and methods are provided for introducing a flowable electrode to a target site within the patient such that the flowable electrode converts to a hardened electrode after being introduced to the target site. An electrical impulse is applied to the hardened electrode to modulate one or more nerve(s) at the target site. The electrode preferably comprises a conductive polymer material.

### CROSS REFERENCE TO RELATED APPLICATIONS

The present application is a continuation-in-part of U.S. patent application Ser. No. 12/246,605 filed Oct. 7, 2008, which in turn claims priority to U.S. patent application Ser. No. 11/735,709, filed Apr. 16, 2007 and U.S. Provisional Patent Application Nos. 60/792,823, filed Apr. 18, 2006 and 60/978,240, filed Oct. 8, 2007. This application is also a continuation-in-part of U.S. patent application Ser. No. 12/422,483 filed Apr. 13, 2009 which in turn claims priority to co-pending U.S. patent application Ser. No. 12/408,131, filed Mar. 20, 2009, the entire disclosure of which is hereby incorporated by reference. This application is also related to commonly assigned co-pending U.S. patent Ser. Nos. 11/555,142, 11/555,170, 11/592,095, 11/591,340, 11/591,768 and 11/754,522, the complete disclosures of which are incorporated herein by reference for all purposes.

### BACKGROUND OF THE INVENTION

The present invention relates to the delivery of electrical energy to bodily tissues for therapeutic purposes and more specifically to the use of electrical energy to modify tissue and/or nerves at a target site within a patient.

The use of electrical stimulation for treatment of medical conditions has been well known in the art for nearly two thousand years. It has been recognized that electrical stimulation of the brain and/or the peripheral nervous system and/or direct stimulation of the malfunctioning tissue, which stimulation is generally a wholly reversible and non-destructive treatment, holds significant promise for the treatment of many ailments.

For many years, electrical stimulation of nervous tissue has been used to control chronic pain or treat other disorders. This therapy originates from an implanted source device, called an electric signal generator. The electrical signals, usually a series of brief duration electrical pulses, are delivered through one or more implanted leads that communicate with the source device, and contain several conductive metal electrodes to act as low impedance pathways for current to pass to tissues of interest. For example, in spinal cord stimulation (SCS) techniques, electrical stimulation is provided to precise parts of the human spinal cord through a lead that is usually deployed in the epidural space dorsal to the spinal cord. Such techniques have proven effective in treating or managing disease and chronic pain conditions.

The use of spinal cord stimulation (SCS) in the management of pain syndromes is a minimally invasive and reversible, implantable neurostimulation modality. This modality has been shown clinically to be effective over a range of maladies including ischemic heart disease—refractory angina pectoris, low back pain with radiculopathy, failed-back surgery syndrome (FBSS), abdominal pain, peripheral vascular disease, and complex regional pain syndrome (CRPS). Reports of SCS clinical success range from 50% to 80% with reductions in medication requirements as well as improvements in pain intensity scores, quality of life (QOL) enhancements, corrected function, and bolstered chances of returning to work.

Spinal cord stimulators typically include one or more electrode leads implanted in the epidural space either percutaneously or by surgical laminectomy or laminotomy. A pulse generator or RF receiver may be implanted, for example in the abdomen or buttocks, to apply an electric impulse to the electrode(s) to block pain signals from reaching the brain such that the patient receives a mild tingling sensation in lieu of the pain.

Percutaneous leads are small diameter leads that may be inserted into the human body through a Tuohy (non-coring) needle, which includes a central lumen through which the lead is guided.

Percutaneous leads are advantageous because they may be inserted into the body with a minimum of trauma to surrounding tissue. On the other hand, the designs of lead structures that may be incorporated into percutaneous leads are limited because the lead diameter or cross-section must be small enough to permit the lead to pass through the Tuohy needle, generally less than 2.0 mm diameter. Typically, the electrodes on percutaneous leads are cylindrical metal structures, with a diameter of approximately 1.0 mm and a length of 4.0 to 10.0 mm. Of course, half of each of these electrodes, facing away from the tissue of interest, is not very useful in delivering therapeutic current. Thus the surface area of electrodes that face the tissue to be excited is small, typically 3.0 to 10.0 square mm.

Ideally, an implantable electrode for tissue stimulation in the spinal cord must have several additional features for use in the human body. For one, substantially large conducting electrodes are needed to safely and reliably pass stimulation electrical pulses of adequate amplitudes to excite tissue cells over indefinitely long periods of time. In addition, to minimize surgical trauma during implantation, the electrodes should assume a one dimensional shape that is very narrow inside the lead body (or sheath) for passage through a small catheter or Tuohy needle, and have the ability to assume a two dimensional shape when outside the lead body. Since there may be considerable deposits of fibrosis or scar tissue around each electrode within a few months of permanent implantation, if necessary, the lead should be able to be removed by gentle traction on the lead body, and have all parts easily disengage from the tissue.

### SUMMARY OF THE INVENTION

The present invention provides systems, apparatus and methods for selectively applying electrical energy to body tissue. More specifically, systems and methods are provided for introducing an electrode in a flowable state (i.e., liquid and/or gel-like) to a target region in the body such that the electrode converts to a hardened or solid state at the target region. Electrical energy is delivered to the electrode in the hardened state to modify tissue and/or nerves at the target region. This allows the surgeon to introduce the electrode to the target region within the patient through a minimally-invasive or percutaneous access port. In addition, the flowable nature of the electrode allows the physician to precisely position the electrode at the target site and thus more effectively treat the patient's ailment.

In one aspect of the invention, a device for delivering electrical energy to a patient includes an electrode comprising an electrically conductive material configured to convert from a flowable state to a hardened state and an introducer configured to introduce the electrode in the flowable state to a target site in the patient. The device further includes an electrical contact sized and shaped for positioning within the electrode in either the hardened or flowable state and a source of electrical energy coupled to the electrical contact for delivering an electrical impulse to the electrode in the hardened state. In one embodiment, the electrode may comprise a material designed to convert to the hardened state at body temperature. In an alternative embodiment, the electrode comprises first and second materials that convert to the hardened state upon contact with each other.

In a preferred embodiment, the electrode comprises a biocompatible conductive polymer, an organic polymer that conducts electricity, such as polyacetylenes, polypyrroles, polythiophenes, polyanilines and poly(p-phenylene vinylenes) (PPV). In certain embodiments, the conductive polymer may include an electrically conductive solution, such as saline, to increase the conductivity of the polymer and ensure that the electrode has a higher electrical conductivity than the surrounding tissue. The high conductivity of the resulting polymer and saline composition makes the entire composition effectively equipotential so that it acts as one large electrode at the target region within the patient.

In certain embodiments, the electrode is designed for an acute treatment or treatments and comprises a resorbable material. The resorbable polymer is designed to resorb into the patient's tissue after the acute treatment(s) have been completed so that it does not have to be removed from the patient. In other embodiments, the electrode comprises a non-degradable material that will remain in place without resorbing or degrading, thereby allowing for permanent implantation of the polymer electrode in the patient.

In certain embodiments, the introducer is configured for introduction through a natural orifice in the patient and/or through a port or access channel in an endoscopic procedure to a target region in the body for electrical stimulation (e.g., the bladder or pelvic floor to treat incontinence). In other embodiments, the introducer comprises a needle configured to inject the electrode in the flowable state to the target site. The needle is preferably sized and shaped for advancement through a percutaneous penetration in the patient's skin to a target region within the body. In one exemplary embodiment, the needle is configured for introduction between first and second vertebral bones into an epidural space of the patient. In another exemplary embodiment, the needle is configured for introduction through a percutaneous penetration in the patient's neck to a target region in or around the carotid sheath and/or vagus nerve. In yet other embodiments, the needle may be configured for advancement to a target region in the patient's brain, joints, bladder and/or peripheral nerves.

In one embodiment, the return electrode is a return pad located on a surface of the patient's skin, such as the back or hip, and the hardened electrode acts as the tissue treatment or active electrode. In alternative embodiments, the return electrode may be located closer to the active electrode in or around the target site. In these embodiments, the electrical energy will not flow completely through the patient's body, i.e., the current will generally flow from the active electrode through the patient's tissue at the target site and to the return electrode.

In a preferred embodiment, the source of electrical energy is an electrical signal generator operating to apply at least one electrical signal to the hardened electrode such that, when the electrode is positioned at the target region within the patient, an electro-magnetic field emanates from the electrode to at least one of nerves and muscles in a vicinity of the target site. The electric signal will of course vary depending on the specific application but typically has a frequency between about 1 Hz to 1000 Hz, more preferably between about 1 Hz to about 200 Hz, a pulse duration between about 10-1000 us, preferably between 100 and 500 us, and an amplitude of between about 0.1 to 30 volts, preferably between 1-12 volts.

In another aspect of the invention, a method for treating an ailment in a patient comprises introducing a flowable electrode to a target site within the patient such that the flowable electrode changes to a hardened electrode after being introduced to the target site and applying an electrical impulse to the hardened electrode to modulate one or more nerve(s) at the target site. In one embodiment, the introducing step is carried out by injecting first and second materials to the target site such that the first and second materials contact each other and convert to the hardened electrode. In an alternative embodiment, the introducing step comprises injecting a flowable material that automatically hardens at body temperature.

In one exemplary embodiment, the flowable electrode is introduced to a target site within an epidural space of the patient such that the hardened electrode contacts a dura within the epidural space. To that end, a needle is advanced through a spinal ligament between first and second vertebral bones and the flowable electrode is injected through the needle directly into the epidural space such that the electrode hardens onto the patient's dura. Thus, the flowable electrode can be injected into the patient's epidural space through a small portal, and then expanded into the hardened state inside the epidural space to achieve a larger footprint of contact on the dura. This substantially prevents migration of the electrode within the epidural space and provides for more efficient and effective treatment.

The method preferably includes applying an electrical impulse to a sympathetic nerve chain of a patient to block, stimulate and/or modulate nerve signals to treat a gastrointestinal disorder of the patient. In this embodiment, an electrical impulse can be applied to increase an intestinal and/or gastric motility of the patient, decrease pain associated with irritable bowel syndrome and/or improve intestinal peristalsis function within the patient.

In an exemplary embodiment, the present invention includes a method of increasing intestinal motility of a patient suffering from post-operative ileus. In this procedure, the flowable electrode of the present invention is introduced through a percutaneous penetration in the patient and advanced to an epidural space between T5 and L2, preferably around T7. The electrode is then hardened to thereby contact an expanded surface area of the dura as described above. An electrical impulse is applied to the hardened electrode; preferably having a frequency between about 10 Hz to 200 Hz, preferably between about 25 to 50 Hz, a pulse duration of between about 20-400 us, and an amplitude of between about 1-20 volts. The impulse modulates one or more nerves around the epidural space to at least partially improve intestinal peristalsis resulting from the operation.

Other aspects, features, advantages, etc. will become apparent to one skilled in the art when the description of the invention herein is taken in conjunction with the accompanying drawings.

### BRIEF DESCRIPTION OF THE DRAWINGS

For the purposes of illustrating the various aspects of the invention, there are shown in the drawings forms that are presently preferred, it being understood, however, that the invention is not limited by or to the precise arrangements and instrumentalities shown.

FIG. 1 schematically illustrates an exemplary electrical stimulation system according to the present invention;

FIG. 2 illustrates an electrode lead and contact according to one embodiment of the invention;

FIG. 3 illustrates an introducer according to one embodiment of the present invention;

FIG. 4 illustrates a method of using the electrical stimulation system of FIG. 1 to modulate one or more nerve(s) in or around the carotid sheath;

FIG. 5 illustrates the introducer of FIG. 3 as it is advanced through a percutaneous penetration in a patient to the target region near the carotid sheath;

FIG. 6 illustrates the electrode lead and contact of FIG. 2 as it is advanced through the introducer to the target region in the patient;

FIG. 7 illustrates an exemplary connector for coupling the electrode assembly of FIG. 2 to a source of electrical energy (not shown);

FIG. 8 illustrates removal of the introducer and the electrode assembly and connector after said removal, respectively; and

FIG. 9 is a perspective view of the electrode lead and introducer of the present invention being advanced to a target location within the spinal cord according to the present invention.

### DETAILED DESCRIPTION OF THE PREFERRED EMBODIMENTS

In the present invention, electrical energy is applied to one or more electrodes to deliver an electromagnetic field to a patient. The invention is particularly useful for applying electrical impulses that interact with the signals of one or more nerves or muscles to achieve a therapeutic result, such as treating bladder incontinence, epilepsy, depression, Parkinson's disease, stroke, schizophrenia, multiple sclerosis, neuralgia, the relaxation of the smooth muscle of the bronchia to treat asthma, anaphylaxis or COPD, the increase in blood pressure to treat orthostatic hypotension, sepsis or hypovolemia, Crohn's disease, obesity, sleep apnea, type 1 or 2 diabetes, treating ischemic heart disease—refractory angina pectoris, congestive heart failure, low back pain with radiculopathy, failed-back surgery syndrome (FBSS), abdominal pain, peripheral vascular disease, complex regional pain syndrome, treating ileus conditions, IBS, and/or any other ailment affected by nerve transmissions. In addition, the present invention can be used to practice the treatments described in the following commonly assigned patent applications: US Patent Publication Numbers: 2009/0183237, 2008/0009913, 2007/0191902, 2007/0191905, 2007/0106339, 2007/0106338 and 2007/0106337, the full disclosures of which were previously incorporated herein by reference.

For convenience, the remaining disclosure will be directed specifically to the treatment of nerves in or around the carotid sheath and within the spinal cord with a device introduced through a percutaneous penetration in the patient, but it will be appreciated by those skilled in the art that the systems and methods of the present invention can be applied equally well to other tissues and nerves of the body, including but not limited to other parasympathetic nerves, sympathetic nerves, spinal or cranial nerves, e.g., optic nerve, facial nerves, enteric nerves, vestibulocochlear nerves and the like. In addition, the present invention can be applied in other procedures including open procedures, intravascular procedures, interventional cardiology procedures, urology, laparoscopy, general surgery, arthroscopy, thoracoscopy or other cardiac procedures, cosmetic surgery, orthopedics, gynecology, otorhinolaryngology, spinal and neurologic procedures, oncology procedures and the like.

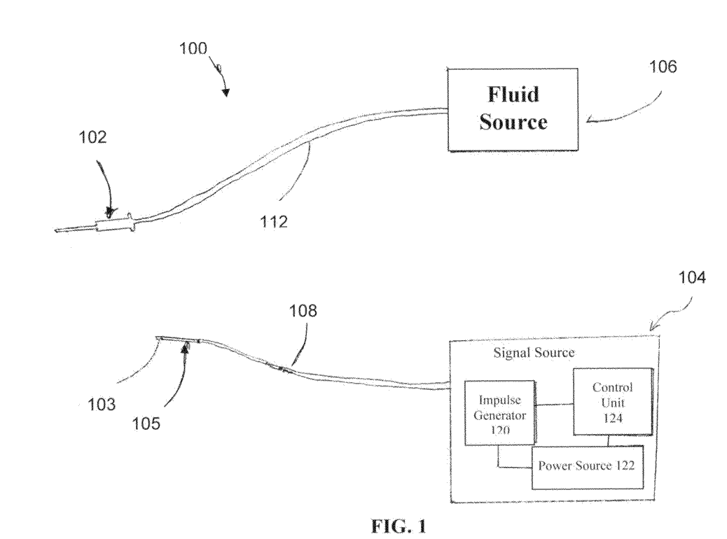

Referring to the drawings in detail, wherein like numerals indicate like elements, FIG. 1 schematically illustrates an exemplary electrical stimulation system 100 according to the present invention. System 100 comprises an introducer 102 and an electrical contact 103 at the distal end of an electrode lead 105. The electrode lead 105 is coupled to an electrical signal generator or source 104 for providing an electrical impulse to a target tissue. Electrode lead 105 includes an elongated shaft or connector 108 which may be flexible or rigid, with flexible shafts optionally including support cannulas or other structures (not shown). In a preferred embodiment, lead shaft 108 is a thin insulated wire having a small electrode contact 103 at its distal tip. In a preferred embodiment, introducer 102 comprises a needle such as a syringe designed for injecting a fluid or gel-like material through a percutaneous penetration in the patient. System 100 further comprises a fluid source 106 for injecting a flowable conductive polymer (not shown) through introducer 102 to a target site within the patient.

A conductive fluid (not shown) such as saline may also be introduced through fluid tube 112 (or through a second fluid tube not shown) to mix with the polymer electrode as it hardens at the target site. The electrical properties of the hardened electrode (with or without the conductive fluid) is preferably designed such that a resistance therethrough is no more than about 1000 Ohms, preferably no more than 500 Ohms and more preferably 200 Ohms or less. The electrically conducting fluid should have a threshold conductivity to provide a suitable conductive path between electrical contact 103 and through the electrode to the tissue at the target site. To that end, the electrical conductivity of the fluid (in units of milliSiemans per centimeter or mS/cm) will typically be between about 1 mS/cm and 200 mS/cm and will usually be greater than 10 mS/cm, preferably will be greater than 20 mS/cm and more preferably greater than 50 mS/cm. In one embodiment, the electrically conductive fluid is isotonic saline, which has a conductivity of about 17 mS/cm. Applicant has found that a more conductive fluid, or one with a higher ionic concentration, will usually provide optimal results. For example, a saline solution with higher levels of sodium chloride than conventional saline (which is on the order of about 0.9% sodium chloride) e.g., on the order of greater than 1% or between about 3% and 20%, may be desirable. A fluid of about 5% saline (e.g., approximately 100 mS/cm) is believed to work well, although modifications to the concentration and the chemical make-up of the fluid may be determined through simple experimentation by skilled artisans.

In an alternative embodiment, system 100 includes at least two fluid sources (not shown) coupled to the distal end of introducer 102 or to two different introducers. In this embodiment, at least two separate flowable materials are injected from the multiple fluid sources to the target site. The flowable materials are designed to harden into an electrode upon contact with each other.

System 100 may also include a return electrode (not shown) adapted for placement on the outer surface of the patient's skin (e.g., the back or buttocks) such that the electrical current passes through the target site and the patient's body to the return electrode. Alternatively, a second electrode having the opposite polarity as the flowable electrode may be positioned near or adjacent to contact 103 such that the electrical current is confined to the target site. The second electrode may optionally be a flowable conductive polymer material that is also injected into the target site.

Electrical source 104 operates to apply at least one electrical signal to contact 103 such that, when contact 103 is positioned at a target site in a patient (such as the spinal cord or the carotid sheath) and the flowable electrode hardens (described below), an electro-magnetic field emanates from the electrode to the anatomy of the mammal in the vicinity of the target site to achieve a therapeutic result. Electrical source 104 may be tailored for the treatment of a particular ailment and may include an electrical impulse generator 120, a power source 122 coupled to the electrical impulse generator 120, and a control unit 124 in communication with the electrical impulse generator 120 and the power source 122. The electrodes provide source and return paths for the at least one electrical signal to/from the contact 103 and the return electrode (which is either located near contact 103 or elsewhere as discussed above). The control unit 124 may control the electrical impulse generator 120 for generation of the signal suitable for amelioration of the ailment when the signal is applied to the electrical contact 103. It is noted that source 104 may be referred to by its function as a pulse generator.

A suitable electrical voltage/current profile for the stimulating, blocking and/or modulating impulse to the portion or portions of one or more nerves and/or muscles may be achieved using the pulse generator 120. In a preferred embodiment, the pulse generator 120 may be implemented using the power source 122 and control unit 124 having, for instance, a processor, a clock, a memory, etc., to produce a pulse train to the electrode(s) that deliver the blocking and/or modulating fields to the nerve resulting from the electrical impulses.

The parameters of the modulation signal are preferably programmable, such as the frequency, amplitude, duty cycle, pulse width, pulse shape, etc. The impulse signal preferably has a frequency, an amplitude, a duty cycle, a pulse width, a pulse shape, etc. selected to influence the therapeutic result, such as stimulating, blocking and/or modulating some or all of one or more nerve transmissions. Assuming the aforementioned impedance characteristics of the device 100, the at least one electrical signal may be of a frequency between about 1 Hz to 3000 Hz, a pulse duration of between about 10-1000 us, and an amplitude of between about 1-20 volts. For example, for treating post-operative ileus (discussed below), the electrical signal may be of a frequency between about 15 Hz to 35 Hz, such as about 25 Hz. The at least one electrical signal may have a pulsed on-time of between about 50 to 1000 microseconds, such as between about 100 to 300 microseconds, such as about 200 microseconds. The at least one electrical signal may have an amplitude of about 1-15 volts, such as about 8-12 volts. The at least one electrical signal may include one or more of a full or partial sinusoid, a square wave, a rectangular wave, and triangle wave.

Although the specific implementation of the signal source is not of criticality to the invention, by way of example, the source may be purchased commercially, such as a Model 7432 available from Medtronic, Inc. Alternatively, U.S. Patent Application Publications 2005/0075701 and 2005/0075702, both to Shafer, both of which are incorporated herein by reference, contain descriptions of pulse generators that may be applicable for implementing the signal source of the present invention.

An alternative implementation for the signal source of the present invention may be obtained from the disclosure of U.S. Patent Publication No.: 2005/0216062, the entire disclosure of which is incorporated herein by reference. U.S. Patent Publication No.: 2005/0216062 discloses a multi-functional electrical stimulation (ES) system adapted to yield output signals for effecting faradic, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated.

In use, introducer needle 102 is advanced through a percutaneous penetration in the patient to a target region in or around the target nerves within the patient (e.g., such as a location within the epidural space or around the vagus nerve in the patient's neck). Electrode lead 105 and contact 103 are then advanced through needle 102 to the target site and a flowable polymer material is delivered from fluid source 106 through fluid tube 112 and needle 102 to the target site. Alternatively, lead 105 and contact 103 can be advanced to the target site outside of needle 102 before or after the conductive polymer has been injected. In either event, the contact 103 is placed within the polymer material before it completely hardens to provide a conductive path from electrical source 104 to the hardened polymer.

In some embodiments, the conductive polymer electrode will harden at body temperature so that it starts to harden as it leaves the tip of the needle. The hardened electrode will typically conform to an area of target tissue (such as the dura) that is larger than the size of the percutaneous penetration. This allows the physician to stimulate a much larger target area than would otherwise be possible through a percutaneous procedure. In addition, the injection of the polymer allows for precise positioning of the electrode to more effectively treat the patient's ailment.

In other embodiments, two or more flowable components will be injected together to the target site such that they harden upon mixing with each other. In both embodiments, a conductive fluid will also be injected to the target site before the electrode is completely hardened so that the combined electrode/fluid composition becomes effectively equipotential and acts as one large electrode.

Conductive polymers are organic polymers that conduct electricity. Such compounds may be true metallic conductors or semiconductors. It is generally accepted that metals conduct electricity well and that organic compounds are insulating, but this class of materials combines the properties of both. The biggest advantage of conductive polymers is their processability. Conductive polymers are also plastics (which are organic polymers) and therefore can combine the mechanical properties (flexibility, toughness, malleability, elasticity, etc.) of plastics with high electrical conductivities. Their properties can be fine-tuned using the exquisite methods of organic synthesis.

In traditional polymers such as polyethylenes, the valence electrons are bound in sp³ hybridized covalent bonds. Such “sigma-bonding electrons” have low mobility and do not contribute to the electrical conductivity of the material. The situation is completely different in conjugated materials. Conducting polymers have backbones of contiguous sp² hybridized carbon centers. One valence electron on each center resides in a p_{z} orbital, which is orthogonal to the other three sigma-bonds. The electrons in these delocalized orbitals have high mobility, when the material is “doped” by oxidation, which removes some of these delocalized electrons. Thus the p-orbitals form a band, and the electrons within this band become mobile when it is partially emptied. In principle, these same materials can be doped by reduction, which adds electrons to an otherwise unfilled band. In practice, most organic conductors are doped oxidatively to give p-type materials. The redox doping of organic conductors is analogous to the doping of silicon semiconductors, whereby a small fraction silicon atoms are replaced by electron-rich (e.g., phosphorus) or electron-poor (e.g. boron) atoms to create n-type and p-type semiconductors, respectively.

Well-studied classes of organic conductive polymers include poly(acetylene)s, poly(pyrrole)s, poly(thiophene)s, polyanilines, polythiophenes, poly(p-phenylene sulfide), and poly(p-phenylene vinylene)s (PPV). PPV and its soluble derivatives have emerged as the prototypical electroluminescent semiconducting polymers. Other less well studied conductive polymers include polyindole, polypyrene, polycarbazole, polyazulene, polyazepine, poly(fluorene)s, and polynaphthalene.

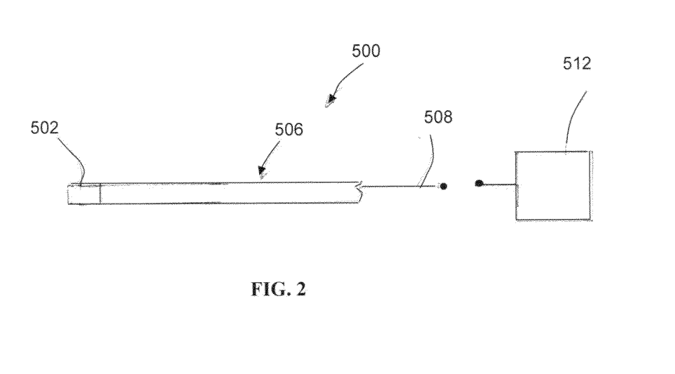

FIG. 2 illustrates an exemplary electrode lead assembly 500 according to one exemplary embodiment of the present invention. As shown, electrode lead assembly 500 includes an active electrical contact 502 coupled to the distal end of an insulating flexible shaft 506. The active electrical contact 502 has a lead 508 extending through shaft 506 for coupling the electrodes to a connector block 512 proximal to the shaft 506. Although there are a number of sizes and shapes that would suffice to implement electrical contact 502, by way of example, contact 502 may be between about 0.5 mm to 5 mm long and may have an outside diameter of between about 0.1 mm to 1 mm. A suitable electrode may be formed from Pt—IR (90%/10%), although other materials or combinations or materials may be used, such as platinum, tungsten, gold, copper, palladium, silver or the like.

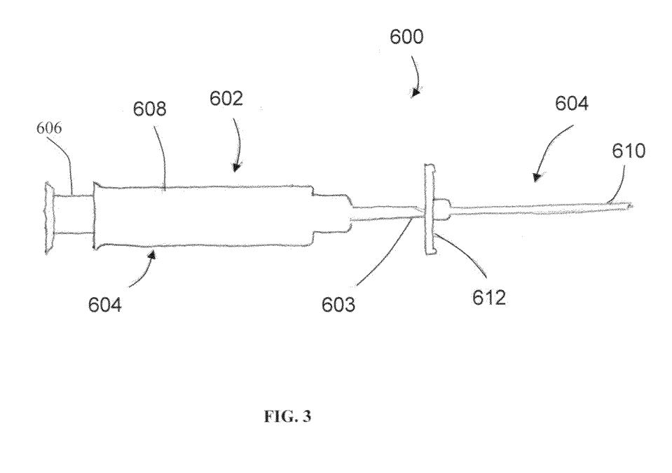

FIG. 3 illustrates an exemplary introducer 600 according to one embodiment of the present invention. As shown, introducer 600 includes a needle assembly 602 and a sheath or cannula 602. In this embodiment, needle assembly 602 is a syringe having a hypodermic needle 603 coupled to a piston pump 604 with a plunger 606 that fits within a cylindrical hollow tube 608. As is well known in the art, plunger 606 can be pulled and pushed along the inside of tube to take in and expel liquids or gases through an orifice (not shown) at the open end of tube 608. Cannula 602 includes a base 612 and a hollow tube 610 sized to receive hypodermic needle 603 and electrode assembly 500 (as discussed below). Although the specific cannula used is not of criticality to the invention, suitable cannulas can be purchased commercially from Epimed.

Alternatively, the introducer may comprise a cannula, trocar, Crawford needle or other hollow access tube that allows for percutaneous or minimally invasive access to a target site within the patient. The flowable polymer may be advanced through the hollow tube by pressure, gravity or by injecting the material into the proximal end of the tube with a syringe or the like.

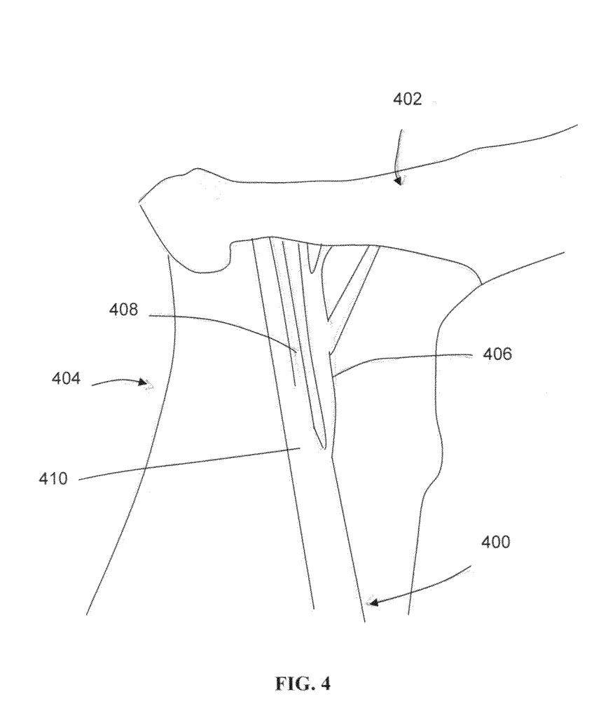

FIGS. 4-8 illustrate an exemplary method of modulating one or more nerves in or around the carotid sheath with the electrical stimulation system of the present invention. In certain embodiments, a flowable polymer is injected in or around the carotid sheath to modulate nerves such as the vagus nerve to treat various ailments. Referring now to FIG. 4, the common carotid artery 400 extends from the base of the skull 402 through the neck 404 to the first rib and sternum (not shown). Carotid artery 400 includes an external carotid artery 406 and an internal carotid artery 408 and is protected by fibrous connective tissue called the carotid sheath. The carotid sheath is located at the lateral boundary of the retopharyngeal space at the level of the oropharynx on each side of the neck 404 and deep to the sternocleidomastoid muscle. The three major structures within the carotid sheath are the common carotid artery 400, the internal jugular vein 410 and the vagus nerve (not shown). The carotid artery lies medial to the internal jugular vein and the vagus nerve is situated posteriorly between the two vessels.

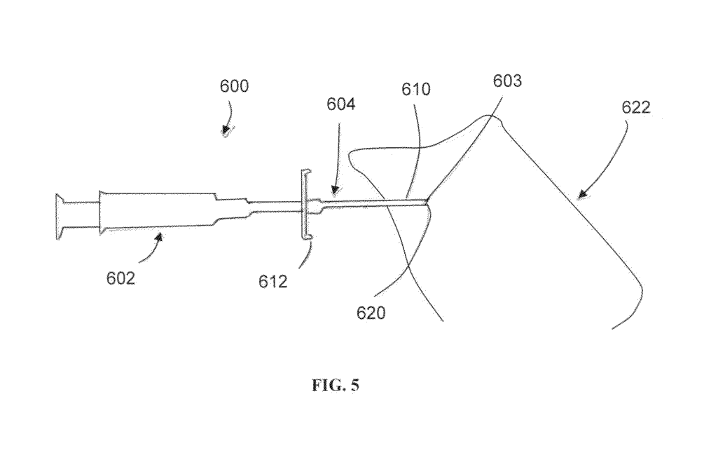

FIGS. 5-8 illustrate a method of applying an electrical impulse to the carotid sheath of a patient according to the present invention. Typically, the carotid sheath or jugular vein will be located in any manner known in the art, e.g., by feel or ultrasound. Once the patient is prepared for the procedure, the target area of the skin on the neck is anesthetized (e.g., with lidocaine or a similar local anesthesia). The target area may be any suitable location that will allow for access to the carotid sheath.

In one embodiment, a finder needle (not shown) may be used to first locate the target region around the carotid sheath. The finder needle is preferably a small access needle having a size in the range of 18-26 gauge, preferably around 22 gauge. Suitable finder needles for use in the present invention may be purchased commercially from Epimed. Typically, the finder needle is inserted through the skin surface and advanced to approach the carotid sheath. In certain embodiments, nerves extending through the carotid sheath, such as the vagus nerve, are targeted for modulation. An excitable tissue cell, such as a nerve fiber, is substantially less sensitive to a transverse electric field than a longitudinal electric field. Applying a longitudinal field increases the effect of this field on the excitable cell at the same frequencies, amplitudes, pulse durations and power levels. Thus, in these embodiments, the finder needle is preferably advanced to approach the carotid sheath in parallel. In other embodiments, the finder needle may be advanced to positions transverse to the carotid sheath.

The finder needle may be aspirated at this point to ensure that it has not penetrated the jugular vein or carotid artery. Alternatively, ultrasound may be used to verify the exact placement of the finder needle. Once the finder needle is in place, an additional incision may be made, e.g. with a scalpel, to provide access to introducer 600. In alternative embodiments, introducer 600 may be directly inserted into patient without the use of a finder needle as described above. As shown in FIG. 5, tube 610 of introducer 600 is driven through a percutaneous penetration 620 in the neck 622 of patient and advanced along the same entry path as the finder needle until it reaches the desired depth of placement of the target region proximal to the carotid sheath. The physician may also aspirate needle 603 to ensure that it has not penetrated into a venous or arterial structure.

Once the distal tip of needle 603 has been advanced to the target site in the patient, a flowable conductive polymer (not shown but described previously) is injected through syringe 602 to the target site. As described above, the polymer can be designed to harden at body temperature so that the polymer hardens soon after exiting needle 602. Alternatively, two compositions can be injected through syringe 602 that harden upon contact with each other after exiting needle 603. In the preferred embodiment, a conductive fluid such as saline will also be injected through syringe either simultaneously with the polymer or immediately thereafter before the polymer completely hardens.

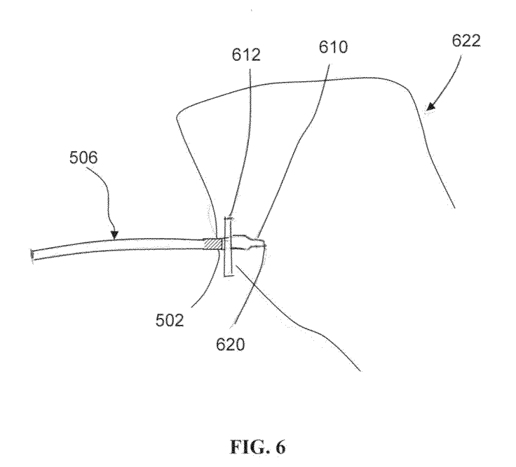

Referring now to FIG. 6, electrode lead 506 and electrical contact 502 may now be inserted into tube 610 and advanced to the target region within the patient. Alternatively, lead 506 and contact 502 may be inserted through syringe 602 before the flowable electrode has been injected into the patient. This ensures that contact 502 is in place at the target site and within the flowable electrode before the polymer hardens. Needle assembly 602 is then removed from cannula 603 by pressing against base 612 while needle assembly 602 is withdrawn.

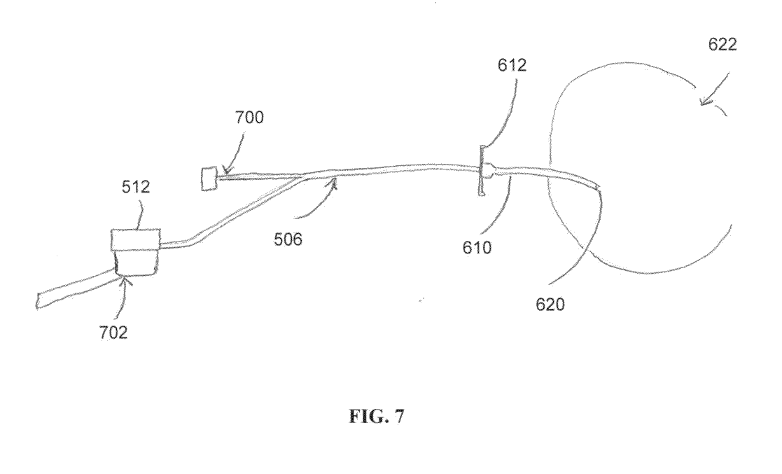

As shown in FIG. 7, once contact 502 is in place within the hardened polymer, connector block 512 is attached to a cable 702 to electrically couple electrical contact 502 to a source of electrical energy (not shown). At this point, the system may be tested to ensure proper functioning by activating the source of electrical energy and noting any muscle tremor at the target region.

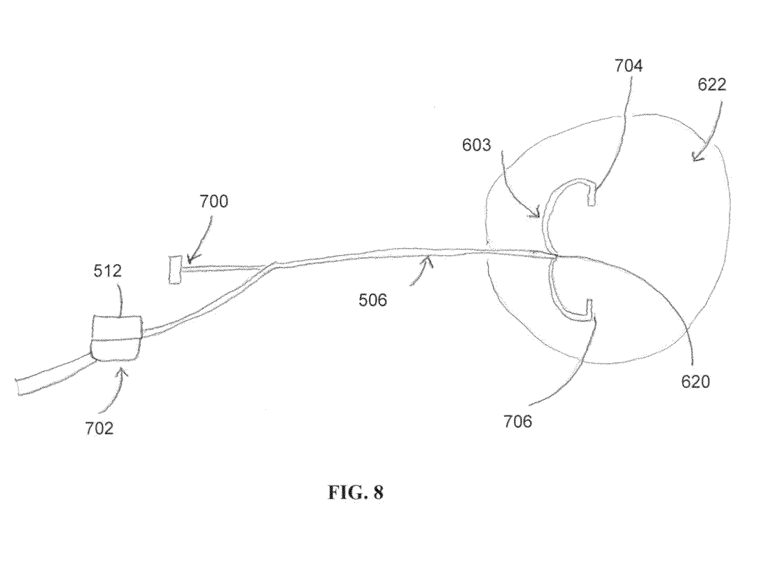

As shown in FIG. 8, cannula 603 may now be removed from the patient. In one embodiment, this is accomplished by bending tabs 704, 706 of base 612 downward and pulling them apart, thereby splitting cannula 603 into two pieces. Cannula 603 is then removed while electrode assembly 500 is held securely to prevent migration during cannula 603 removal. Similarly, delivery stylet 700 may be removed from patient leaving only the electrode assembly 500 in position at the target region. Electrode assembly 500 is then secured in place on the patient, e.g., with the use of tape or sutures (not shown), to ensure that it does not migrate during the procedure. Alternatively, stylet 700 and/or cannula 603 may be left in place during the entire procedure.

In one specific embodiment, method and devices of the present invention are particularly useful for providing substantially immediate relief of acute symptoms associated with bronchial constriction such as asthma attacks, COPD exacerbations and/or anaphylactic reactions. One of the key advantages of the present invention is the ability to provide almost immediate dilation of the bronchial smooth muscle in patients suffering from acute bronchoconstriction, opening the patient's airways and allowing them to breathe and more quickly recover from an acute episode (i.e., a relatively rapid onset of symptoms that are typically not prolonged or chronic). A more complete description of this procedure can be found in commonly-assigned co-pending U.S. patent application Ser. No. 12/422,483 filed on Apr. 13, 2009, which is incorporated herein by reference.

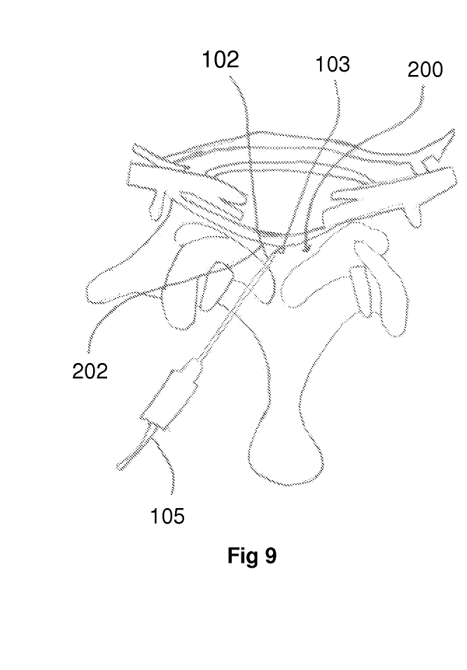

FIG. 9 illustrates a method of modulating nerves within the epidural space 200 of a patient with the nerve stimulation system 100 of the present invention. In use, introducer needle 102 is introduced into the patient as described above such that its distal tip is adjacent to or in contact with a target area within the epidural space 200, such as the dura 202. The target area will of course vary depending on the application. In certain embodiments such as for treating post-operative ileus (described in more detail below), the target area will be between T5 to L2, preferably around T6 or T7.

Once the introducer 102 is in position, electrode lead 105 is advanced through introducer 102 to the target site such that electrical contact 103 can be positioned at the target site within the epidural space 200. A polymer and a conductive fluid, such as saline, are then delivered through fluid tube 112 and introducer 102 to the target site, where they will harden into a large electrode (not shown) around contact 103. An electrical impulse is then generated by signal source 104 and applied to electrical contact 102 to modulate nerves and/or muscles at the target region.

In certain embodiments for treatment of chronic pain, electrical contact 102 and the conductive polymer will be implanted within epidural space 200 and pulse generator 120 may be implanted, for example in the abdomen or buttocks, to apply electric impulse(s) to the electrode. In such embodiments, the electrical impulse may be selected to block pain signals from reaching the brain such that the patient receives a mild tingling sensation in lieu of pain. In other embodiments such as treating post-operative ileus (described in detail below), electrical contact 103 and the conductive polymer may be used acutely for a period of time (e.g., from minutes to days) and then withdrawn from the patient (i.e., without permanently implanting lead 105 or pulse generator 120). In this embodiment, the polymer may comprise a resorbable material that resorbs into the surrounding tissue after a certain period of time such that there is no requirement to remove the polymer from the patient's body.

In another embodiment, the present invention may be used for treating gastrointestinal disorders, such as pain associated with IBS and/or gastric or intestinal motility disorders. In an exemplary embodiment, the present invention describes a method for reversing the temporary arrest of intestinal peristalsis as described more fully in commonly assigned U.S. patent application Ser. No. 12/246,605, which has already been incorporated herein by reference. Recent reviews in the art have discussed the potential application of electrical stimulation of the end organ, namely the stomach, small intestine or colon to improve motility. SCS may also be a useful treatment modality for dysmotility, particularly delayed gastric and intestinal motility following surgery.

In this embodiment, an electrode as described above is introduced into the patient and placed in contact with, or close proximity to, at least one of the celiac ganglia, cervical ganglia and thoracic ganglia of the sympathetic nerve chain. An electric signal is applied to the electrode to induce at least one of an electric current, an electric field and an electromagnetic field in the sympathetic nerve chain to modulate and/or block inhibitory nerve signals thereof such that intestinal peristalsis function is at least partially improved. Alternatively or additionally, the electric current, electric field and/or electromagnetic field may be applied to at least a portion of the splancnic nerves of the sympathetic nerve chain, and/or the spinal levels from T5 to L2.

The electrode may be introduced into the epidural space of the patient after the surgery has been completed. As described more fully above, the flowable electrode is preferably introduced through a small portal and then expanded inside the epidural space as it hardens to achieve a larger footprint of contact on the dura. This ensures that the electric impulse will target the selected nerves to sufficiently influence the therapeutic result. In addition, it inhibits migration of the electrode within the epidural space and provides for a more efficient and effective treatment.

As described more fully in the patent application Ser. No. 12/246,605, drive signals may be applied to the one or more electrodes to produce the at least one impulse and induce the current and/or field(s). The drive signals may include at least one of sine waves, square waves, triangle waves, exponential waves, and complex impulses. The drive signals inducing the current and/or fields preferably have a frequency, an amplitude, a duty cycle, a pulse width, a pulse shape, etc. selected to influence the therapeutic result, namely modulating some or all of the nerve transmissions in the sympathetic nerve chain. By way of example, the parameters of the drive signal may include a square wave profile having a frequency of about 10 Hz or greater, such as between about 15 Hz to 200 Hz, and more preferably between about 15 Hz to about 50 Hz. The drive signal may include a duty cycle of between about 1 to 100%. The drive signal may have a pulse width selected to influence the therapeutic result, such as about 20 us or greater, such as about 20 us to about 1000 us. The drive signal may have a peak voltage amplitude selected to influence the therapeutic result, such as about 0.2 volts or greater, such as about 0.2 volts to about 20 volts.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A device for delivering electrical energy to a patient, comprising:
an electrode comprising an electrically conductive material configured to convert from a flowable state to a hardened state;
an introducer configured to introduce the electrode in the flowable state to a target site in the patient; and
a source of electrical energy coupled to the electrode for delivering an electrical impulse to the electrode in the hardened state.

2. The device of claim 1 wherein the electrode is configured to convert to the hardened state at body temperature.

3. The device of claim 1 wherein the electrode comprises first and second materials and is configured to convert to the hardened state upon contact between the first and second materials.

4. The device of claim 1 wherein the electrode comprises a conductive polymer.

5. The device of claim 4 wherein the conductive polymer comprises an electrically conductive solution.

6. The device of claim 1 wherein the introducer comprises a needle configured to inject the electrode in the flowable state to the target site.

7. The device of claim 6 wherein the needle is configured for advancement between first and second vertebral bones into an epidural space of the patient.

8. The device of claim 1 further comprising an electrical connector for coupling the source of electrical energy to the electrode.

9. The device of claim 1 wherein the electrode comprises a resorbable material.

10. The device of claim 1 wherein the electrode comprises a nondegradable material configured for implantation in the patient.

11. The device of claim 1 wherein the electrode is sized and shaped in the hardened state to conform to a target region in the epidural space of the patient.

12. The device of claim 1 wherein the electrode is sized and shaped in the hardened state to conform to a target region on a nerve.

13. The device of claim 1 wherein the electrode is sized and shaped in the hardened state to conform to a target region on a vagus nerve.

14. The device of claim 1 wherein the introducer is configured to inject the electrode in the flowable state through a percutaneous penetration in the patient.

15. The device of claim 1 wherein the source of electrical energy is an electrical signal generator operating to apply at least one electrical signal to the electrode, the electrical signal having a frequency between about 1 Hz to 3000 Hz, a pulse duration of between about 10-1000 us, and an amplitude of between about 1-20 volts.

16. The device of claim 1 further comprising an electrical contact sized and shaped for positioning within the electrode.

17. A method for treating an ailment in a patient comprising:
introducing a flowable electrode to a target site within the patient such that the flowable electrode changes to a hardened electrode after being introduced to the target site; and
applying an electrical impulse to the hardened electrode to modulate one or more nerve(s) at the target site.

18. The method of claim 17 wherein the introducing step is carried out by injecting first and second materials to the target site such that the first and second materials contact each other and convert to the hardened electrode.

19. The method of claim 17 wherein the introducing step comprises injecting a flowable material that hardens at body temperature to the target site.

20. The method of claim 17 wherein the introducing step comprises injecting the flowable electrode through a percutaneous penetration in the patient.

21. The method of claim 17 further comprising electrically coupling the hardened electrode to a source of electrical energy.

22. The method of claim 21 wherein the electrically coupling step is carried out by positioning an electrical contact within the flowable electrode at the target site before the flowable electrode changes to the hardened electrode.

23. The method of claim 17 wherein the introducing step comprises introducing the flowable electrode to a target site within an epidural space of the patient such that the hardened electrode contacts a dura within the epidural space.

24. The method of claim 17 wherein the introducing step comprises advancing a needle through a spinal ligament between first and second vertebral bones and injecting the flowable electrode directly into the epidural space.

25. The method of claim 17 wherein the introducing step comprises introducing the flowable electrode to a target site on a nerve within the patient such that the hardened electrode substantially conforms to the nerve.

26. The method of claim 25 wherein the nerve is a vagus nerve.

27. The method of claim 17 further comprising contacting a return electrode to an external portion of the patient and emanating an electro-magnetic field from the hardened electrode through the patient to the return electrode.

28. The method of claim 17 wherein the applying step comprises applying an electrical impulse to a sympathetic nerve of a patient to modulate nerve signals thereof such that intestinal peristalsis function within the patient is at least partially improved.

29. The method of claim 17 wherein the applying step is carried out by applying an electrical signal to the hardened electrode of a frequency between about 10 Hz to 200 Hz, a pulse duration of between about 20-400 us, and an amplitude of between about 1-20 volts.

30. The method of claim 17 wherein the applying step is carried out by applying an electrical impulse to the electrode sufficient to increase an intestinal motility of the patient.

31. The method of claim 17 wherein the applying step is carried out by applying an electrical impulse to the electrode sufficient to increase a gastric motility of the patient.

32. The method of claim 17 wherein the applying step is carried out by applying an electrical impulse to the electrode sufficient to treat pain.

33. The method of claim 32 wherein the pain is visceral pain associated with irritable bowel syndrome.
